(19) United States
(12) Patent Application Publication
(10) Pub. No.: US 2024/0390673 A1
(43) Pub. Date: Nov. 28, 2024

(54) METHODS AND DEVICES FOR EXTENDING GESTATIONAL AGE

(71) Applicant: ExploraMed V, LLC, Mountain View, CA (US)

(72) Inventors: John Chang, Los Altos, CA (US); Joshua Makower, Los Altos Hills, CA (US); Earl Bright II, Sunnyvale, CA (US); James Wall, Woodside, CA (US); John Hanley, Manhattan Beach, CA (US)

(21) Appl. No.: 18/669,236

(22) Filed: May 20, 2024

Related U.S. Application Data
(60) Provisional application No. 63/469,100, filed on May 26, 2023.

(51) Int. Cl. A61N 1/36; A61N 1/05

(52) U.S. Cl. CPC A61N 1/36003; A61N 1/0521

(57) ABSTRACT

Systems and methods for applying an energy means to the uterus to disrupt or suppress the electromechanical status of the uterus. In one aspect, there are provided devices and methods of delaying or suspending preterm labor or birth by applying an energy means or drugs, or other agents to the uterus to disrupt or suppress the electromechanical status or waves of the uterus and render contractions ineffective or block them entirely.

### FIELD OF THE DISCLOSURE

The present disclosure generally relates to systems and methods for extending gestational age of a fetus by delaying birth.

### BACKGROUND OF THE DISCLOSURE

There is a continuing need for safe and effective approaches to delay birth and extend gestational age.

Preterm labor refers to the onset of labor before thirty-seven weeks of pregnancy. Preterm birth is defined as birth before thirty-seven weeks of gestational age. Although most cases do not have an associated risk factor, it can be a significant cause of neonatal morbidity and mortality that can lead to a host of complications including respiratory distress syndrome, bronchopulmonary dysplasia, necrotizing enterocolitis, short gut syndrome, infections, sepsis, intraventricular brain hemorrhage, cerebral palsy, learning disabilities, retinopathy, blindness, and even death of the premature infant.

It is estimated that preterm birth accounts for around 10% of all births worldwide. The exact cause of preterm labor is not well understood. There are several risk factors that have been identified, including a history of preterm birth, infections, multiple pregnancies, smoking, and certain medical conditions like diabetes and high blood pressure. However, most cases occur spontaneously.

Other factors that may contribute to preterm labor include inflammation, stress, and changes in the levels of certain proteins called cytokines, which play a role in the immune response. Infections, such as urinary tract infections, can also trigger preterm labor by causing inflammation and irritation of the uterus.

Increasing gestational age leads to better outcomes and mortality and less morbidity. Preterm babies with higher gestational age are generally better off.

The uterus is made up of muscle tissue, and during labor, it contracts rhythmically to push the fetus out of the womb. The exact trigger for the onset of contractions is not fully understood, but several factors are believed to play a role.

One of the most critical factors is the complex interplay of hormones that regulate pregnancy and birth. For example, the hormone oxytocin plays a crucial role in stimulating uterine contractions during labor. Prostaglandins are another group of hormones that are thought to trigger contractions by softening and thinning the cervix, which is the gateway between the uterus and the birth canal.

Electromechanical factors play an important role in the onset of contractions in the uterus during labor. It has been suggested that the uterus may have a significant number of widely distributed pacemakers, the coordination of which is necessary to the generation of high intrauterine pressures for effective cervical dilation. There is also believed to be a direct relationship between intrauterine pressure and the activity of a pacemaker. If only a portion of a uterus contracts, there is not enough pressure generated for labor. It has been observed that localized activity does not propagate contiguously more than 8-10 cm. It has also been suggested that each pacemaker may generate several action potentials including a plateau type action potential and a burst action potential, and while each contraction can be traced to a particular electrical event, not every contraction involves the same pacemaker.

During pregnancy, the uterus is relatively quiescent due to a balance of electrical and mechanical forces that maintain a state of uterine quiescence. However, during labor, the uterus transitions from a quiescent state to a state of active contractions, which requires a complex interplay between electrical and mechanical factors.

One of the key electromechanical factors that plays a role in the onset of contractions is the activity of ion channels in the myometrium, the smooth muscle tissue of the uterus. These ion channels play a crucial role in the electrical activity of the myometrium, which, in turn, controls the mechanical activity of the uterus. Specifically, changes in the activity of voltage-gated ion channels such as potassium channels and calcium channels have been implicated in the onset of contractions.

Moreover, it has been observed that myocytes propagate action potentials as they contract, but they do not spontaneously generate action potentials on their own. Since the myocytes must express action potentials in order to contract, a pacemaker employing a slow-wave requires depolarization above a threshold of the myocyte. It has been observed that human uterine action potential propagation speeds during labor are on the order of 6-10 cm/s.

In addition, mechanical factors such as stretch and pressure also contribute to the onset of contractions during labor. Notably, myometrial tissue generates contractions and spontaneous electrical activity when subjected to tension. In fact, it is known that high intrauterine pressures are required to dilate the cervix. Thus, as the baby grows and the uterus expands, the pressure and stretch on the uterine wall increase, which can trigger the onset of contractions. Further, contractions increase intrauterine pressure which increases wall tension, which activates pacemakers, which leads to muscle contractions. Additionally, the mechanical stretching of the cervix during labor is thought to play a crucial role in the release of hormones like oxytocin, which stimulate uterine contractions.

Accordingly, the uterine electrical and mechanical mechanisms involve a pacemaker initiating a propagating action potential, which recruits tissue reached by the action potential to thereby create a local contraction. This local contraction increases uterine pressure and the uterine wall stress increases in response as modified by local uterine wall thickness and curvature. Where the additional wall stress is strong enough to initiate pacemaker activity at another location, one or more further pacemakers initiate another local action potential, creating more pressure and wall stress. With multiple local contractions occurring, intrauterine pressure increases even further. The electrical and mechanical processes then repeat thus creating a self-supporting, positive feedback mechanism.

Gap junctions are specialized protein channels that are found in many tissues, including the myometrium of the uterus. These channels play a crucial role in the electrical communication between cells by allowing the passage of ions, second messengers, and small molecules between adjacent cells.

In the myometrium, gap junctions are essential for the coordination of uterine contractions during labor. Gap junctions allow for the rapid spread of electrical signals between cells, which synchronizes the activity of the myometrium and leads to coordinated contractions of the uterus. In this way, gap junctions are involved in facilitating the efficient expulsion of the fetus during labor.

Studies have shown that the expression and function of gap junctions are regulated by various factors, including hormones such as estrogen and progesterone, which play a crucial role in regulating uterine activity. Changes in the expression and function of gap junctions have been implicated in the onset of preterm labor.

Overall, gap junctions play a crucial role in the coordinated activity of the myometrium during labor and are an important target for therapeutic interventions aimed at preventing preterm labor and improving fetal outcomes. Moreover, the existence of gap junctions in the smooth muscle of the uterus implies that the uterus is hardwired electrically and that waves of electromechanical energy can spread across the uterus. These are required for the effective passage of the fetus from the uterus to the birth canal.

Further, it has been recognized that over the course of a pregnancy and during labor, the uterus and its myometrium undergoes significant changes or remodeling. In particular, it has been observed that the number of gap junctions increases. Thus, inherent characteristics of the myometrium can change over time.

Nervous innervation of the uterus can also contribute to uterine contractions. The sympathetic nervous system is often associated with the “fight or flight” response, and its activation can lead to an increase in uterine contractions. Sympathetic nerve fibers release norepinephrine and epinephrine, which bind to beta-adrenergic receptors on the surface of smooth muscle cells in the uterus. This binding triggers a cascade of events that lead to the opening of voltage-gated calcium channels, allowing an influx of calcium ions into the cells and triggering muscle contraction. The sympathetic nervous system also stimulates the release of oxytocin from the posterior pituitary gland, which further enhances uterine contractions.

On the other hand, the parasympathetic nervous system is often associated with the “rest and digest” response, and its activation can lead to a decrease in uterine contractions. Parasympathetic nerve fibers release acetylcholine, which binds to muscarinic receptors on the surface of smooth muscle cells in the uterus. This binding leads to the activation of potassium channels, allowing an efflux of potassium ions out of the cells and hyperpolarization of the cell membrane, which inhibits muscle contraction.

During labor and delivery, the balance between sympathetic and parasympathetic nervous system activity is finely tuned to regulate the frequency, duration, and intensity of uterine contractions. The sympathetic nervous system tends to be more active during the early stages of labor, while the parasympathetic nervous system becomes more dominant during the later stages.

Furthermore, it is known that inhaled anesthetics such as isoflurane and sevoflurane enhance the activity of inhibitory neurotransmitters, such as gamma-aminobutyric acid (GABA), which can lead to a decrease in the release of excitatory neurotransmitters, such as acetylcholine. This decrease in excitatory neurotransmitters can cause smooth muscle cells to become less responsive to stimuli that would normally trigger contractions, resulting in relaxation of the uterus.

In addition, inhaled anesthetics can also affect the levels of intracellular calcium ions in smooth muscle cells, which play a key role in muscle contraction. Anesthetics can decrease the influx of calcium ions into smooth muscle cells by blocking voltage-gated calcium channels or by inhibiting the release of calcium from intracellular stores, leading to decreased uterine contractility.

Accordingly, there is a need for apparatus and methods that provide for extending gestational age, preferably for the effective expulsion of a fetus from the uterus at a target gestational age by affecting or controlling the electrical communications across the uterus. There is also a need for apparatus and methods that anticipate or accommodate the changes in the uterus over time.

The present disclosure addresses these and other needs.

### SUMMARY OF THE DISCLOSURE

Briefly and in general terms, the present disclosure is directed towards systems, devices and methods for extending gestational age by delaying birth. In one aspect, there are provided devices and methods of delaying or suspending preterm labor or birth by applying an energy means or drugs, or other agents to the uterus to disrupt or suppress the electromechanical status or waves of the uterus and render contractions ineffective or block them entirely. There are provided approaches to delay preterm delivery in pregnant women with preterm labor in order to decrease infant morbidity.

In one or more aspects, the methods and devices delay an onset of preterm birth which is associated with less morbidity and higher survival rates, and are temporary in nature, not extending postpartum. The approaches are minimally invasive and maintain the integrity of amniotic membranes. A transabdominal, transvascular and/or vaginal access to the myometrium are employed. Uterine treatments can be short-term or long term.

In one or more embodiments, the disclosed systems, devices and methods are configured to one or more prevent or weaken contraction of the uterus, enhance relaxation of myometrium, or minimize a total area of contraction of the uterus so as to avoid a forceful contraction. Moreover, access to the uterus can be through one or more of the abdomen anteriorly or laterally, the cervix, transvaginally, transvascularly or laterally via minimally invasive surgery.

In other aspects, one or more of the disclosed approaches have effects that are local and restricted to the uterus itself, therefore the safety of the fetus and mother can be maintained without unwanted off-target side effects typical of the drugs used to treat preterm labor.

In one embodiment, one or more electrodes are inserted into the muscle tissues in or in connection with the uterus to introduce an electrical signal directly into the smooth muscle of the uterus. In another embodiment, one or more electrodes are placed on the outer surface of the uterus to introduce an electrical signal into the uterine wall. The electrical signal is capable of spreading across the uterus and is designed to disrupt, block or suppress the effectiveness of the spontaneously generated electromechanical contractions. Certain apparatus are applied in combination with some monitoring or sensing means to deliver energy at specific times to block or disrupt native electromechanical waves.

In various embodiments, electrical stimulation therapy is configured to work in several ways, depending on the specific parameters of the stimulation. In one approach, high-frequency electrical stimulation can be employed to cause depolarization and repolarization of the smooth muscle cells, leading to relaxation. In another approach, low-frequency electrical stimulation can be used to deliver off-timed contractions of the smooth muscle cells resulting in ineffective overall mechanical effectiveness of the uterus and is helpful in stalling the progression of labor. Further, burst electrical stimulation can be utilized to deliver a series of short, high-frequency bursts of electrical energy. This can activate both sensory and motor nerve fibers, leading to inhibition of smooth muscle contractions. Bursts can also be helpful in creating “disruptive” waves causing regions of the uterus to have a high threshold for stimulation at the times when the spontaneous wave is present, thus pausing contractions.

The waveform employed for inhibiting smooth muscle using electrical energy depends on several factors, including the specific condition being treated, the location of the smooth muscle, and the individual patient's needs. The signal or energy employed to inhibit or stimulate the uterus can be configured to change as the uterus or myometrium changes during pregnancy or labor to maintain or optimize effectiveness. In one or more approaches, the amplitude, frequency, period, number of electrodes or other factors can change to achieve the desired effect. Such changes may be automatic in response to sensing changes in the uterus or myometrium or the changes can be pre-programed based on empirical data collected in advance or over time.

Specific delivery tools are provided to insert these electrodes safely on or into the uterus to thereby secure them in the correct location. The electrodes if anchored would include a mechanism to reverse anchoring. In one approach, reversing anchoring can be achieved by applying a sufficient force to extract them, through rotational movements or with the aid of a specific mechanical means for reversing the anchoring means.

In a specific embodiment, patches configured to affect the myometrium are located on the skin. Alternatively, directly contacting or very closely locating apparatus to the myometrium can allow for more effective and efficient delivery of energy to the myometrium. In various approaches, there is contact with the peritoneum (either layer of the parietal or visceral) or the various layers of the uterus (perimetrium, myometrium or even the endometrium).

In one embodiment, multiple electrodes are placed and spaced a given distance apart to ensure greater treatment coverage of the myometrium. The effective regions can overlap in effectiveness region. In another approach, the effective regions do not overlap as any contractions in between regions will be effectively blocked or prevented from propagating past regions of treatment.

Electrodes configured to inhibit contractions can be simple needles, hooks or other conductive members. The electrode design can also allow legs or other features to ‘spread out’ from the initial stick location to ensure further coverage.

In alternative approaches, electromagnetic fields, focused or high energy ultrasound, monopolar or bipolar RF energy, transcutaneous electrical stimulation or thermo-acoustic waves can be employed to create a desirable electrical stimulus or block an existing electrical stimulus. Alternate local delivery methods can also include the administration of pharmaceutical and/or biologic agents. The goal with all such approaches is to avoid significant impact to the fetus.

In alternative approaches, electromagnetic fields, focused or high energy ultrasound, monopolar or bipolar RF energy, transcutaneous electrical stimulation, thermo-acoustic waves, local pharmacological agents, or systemic pharmacological agents can be employed to create a desirable electrical stimulus both invasively and non-invasively to modulate the nervous system remote to the uterus in order to signal and modulate the myometrium to reduce electrical signals and/or muscular contractions.

These and other features of the disclosure will become apparent to those persons skilled in the art upon reading the details of the systems and methods as more fully described below.

### BRIEF DESCRIPTION OF THE DRAWINGS

FIG. 1 is a partial cross-sectional view of a uterus carrying a fetus, depicting an interplay of actions that regulate pregnancy and birth.

FIG. 2 is a partial cross-sectional view, depicting contractions acting on a uterus.

FIG. 3 is a cross-sectional view, depicting stages of remodeling of a uterus.

FIG. 4 is a side view of a uterus, depicting an example of the transmission of contractions along the uterus.

FIG. 5-7 are side views of a uterus, depicting the direction of waves propagating along the uterus.

FIG. 8 is a side view of a uterus, depicting one approach to affecting contractions of the uterus.

FIG. 9 is a side view, depicting an electrode delivery apparatus.

FIG. 10 is a side view, depicting an electrode assembly.

FIG. 11 is a graphical representation, depicting pulsed waveforms.

FIG. 12 is a side view, depicting the generation of disorganized waveforms generated along a uterus.

FIG. 13 is a front view, depicting electrodes attached to a uterus.

FIGS. 14-15 are side views, depicting a uterus with electrodes attached thereto.

FIGS. 16A-B are side views, depicting lines of treatment of a uterus.

FIG. 17-21 are partial side cross-sectional views, depicting various approaches to affecting contractions of a uterus.

FIG. 22A-E are partial front cross-sectional views, depicting transvascular approaches to affecting contractions of a uterus.

FIG. 23 is a top view, depicting a plurality of electrodes positioned for affecting contractions of a uterus.

FIGS. 24-26 are partial side cross-sectional views, depicting yet further approaches to affecting contractions of a uterus.

FIG. 27 is a cross-sectional view, depicting innervation of the uterus and cervix.

FIGS. 28A-B are a cross-sectional view and block diagram, depicting features of another uterine treatment system.

### DETAILED DESCRIPTION OF THE DISCLOSURE

Before the present systems and methods are described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described.

It must be noted that as used herein and in the appended claims, the singular forms “a”, “an”, and “the” include plural referents unless the context clearly dictates otherwise. Thus, for example, reference to “the system” includes reference to one or more systems and equivalents thereof known to those skilled in the art, and so forth.

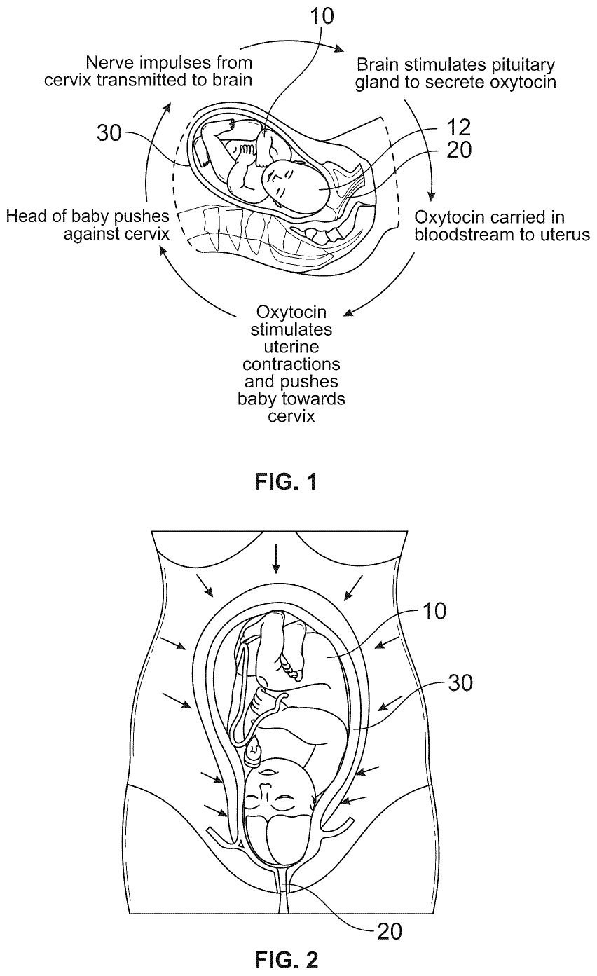

As shown in FIG. 1, a complex interplay of hormones is a critical factor that regulate pregnancy and birth. The hormone oxytocin plays a crucial role in stimulating uterine contractions during labor. As the fetus 10 grows, the head 12 of the fetus 10 pushes against the cervix 20. Nerve impulses from the cervix 20 are transmitted to the brain and the brain then stimulates the pituitary gland to secrete oxytocin. The oxytocin is carried by the bloodstream to the uterus and stimulates uterine contractions, thereby pushing the fetus 10 toward the cervix 20. This consequently causes further nerve impulses to be transmitted to generate further contractions. Prostaglandins are another group of hormones that are thought to trigger contractions by softening and thinning the cervix 20.

Notably, it has been shown that the expression and function of gap junctions found in the myometrium of the uterus are regulated by estrogen and progesterone, which also play a crucial role in regulating uterine activity. As stated, changes in the expression and function of gap junctions have been implicated in the onset of preterm labor.

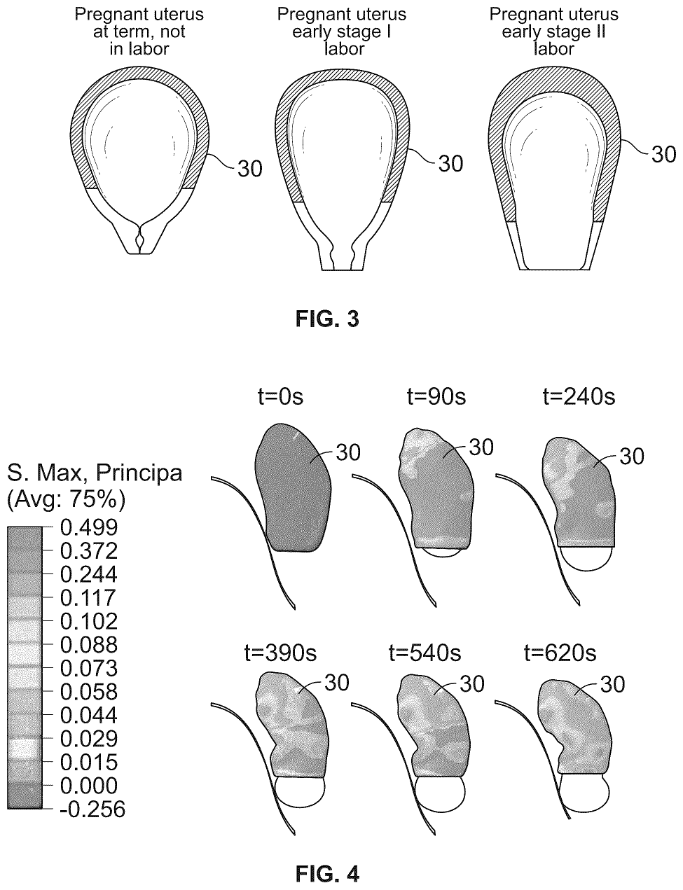

With reference to FIGS. 2-3, as the contractions of the uterus 30 take place, forces are created to facilitate advancing the fetus 10 out of the uterus 30. As this occurs, the uterus 30 remodels about the fetus to create a path for the fetus to advance through the cervix 20 during labor. However, preterm labor is to be avoided as it is associated with significant neonatal morbidity and mortality.

Accordingly, a goal is to provide apparatus and methods that extend gestational age. In particular, one goal is to extend gestational age by delaying preterm labor and subsequent preterm birth. The potential routes to this include one or more of: preventing contractions of the uterus; reducing the effectiveness/strength/efficiency of uterine contractions; inducing disorganized contractions which reduce overall effectiveness/efficiency; reducing an area of uterus that is contracting; enhancing relaxation of the uterus; and/or utilizing a device that delivers energy, such as electrical stimulation, to the myometrium of the uterus which in turn prevents the smooth muscle cells of the uterus to contract or contract less effectively.

In view of the changes to or remodeling of the uterus and its myometrium that occur over the course of a pregnancy and during labor including an increase in the number of gap junctions increases, the signal or energy employed to inhibit or stimulate the uterus can be configured to accommodate or be responsive to such changes to maintain or optimize effectiveness. In one or more embodiments, such changes may be automatic in response to sensing changes in the uterus or myometrium or the changes can be pre-programed based on empirical data collected in advance or over time. In various aspects, the amplitude, frequency, period or other factors can change to achieve the desired effect. Such changes can also be patient or healthcare provider initiated. In particular, a patient can initiate operation of the uterine treatment device, such as by pushing an activation button or otherwise initiate a sequence of inhibition, when they sense a contraction or another cue of an impending contraction. Here, AI/machine learning can be employed to understand sensor readings at the time of patient initiation to then potentially suggest or auto-initiate the uterine treatment device.

In various embodiments, there are provided approaches to delay preterm delivery in pregnant women with preterm labor in order to decrease infant morbidity. In one or more approaches, the disclosed systems, devices or methods delay delivery by up to or more than two weeks, have minimal or no detrimental effect on the fetus, are associated with minimal increase in chorioamnionitis, and are usable by maternal fetal medicine specialists. In other approaches, the disclosed systems, devices or methods delay delivery to full term or at least thirty-four or thirty-seven weeks of gestational age, enable future pregnancies, have associated therewith no increase in chorioamnionitis and are usable by a general obstetrician. Further, such systems, devices and methods have minimal lost constraints due to hepatitis E virus (HEV) that can be common due to immunologic changes during pregnancy that promote the maintenance of the fetus in the maternal environment by the suppression of T-cell mediated immunity.

As shown in FIG. 4, over time, contractions propagate along the uterus 30 (represented by changes in shading) and can involve various and variable sections of the uterus 30 during labor (Pouca et al.; Simulation of the uterine contradictions and foetus expulsion using a chemo-mechanical constitutive model (Biomechanics and Modeling in Mechanobiology, 2019)). Therefore, as myometrial contraction propagation may be limited in distance direction or area, it may be preferred to deliver energy to multiple locations of the myometrium to affect a large enough total area of the uterus to minimize the effectiveness of any contraction.

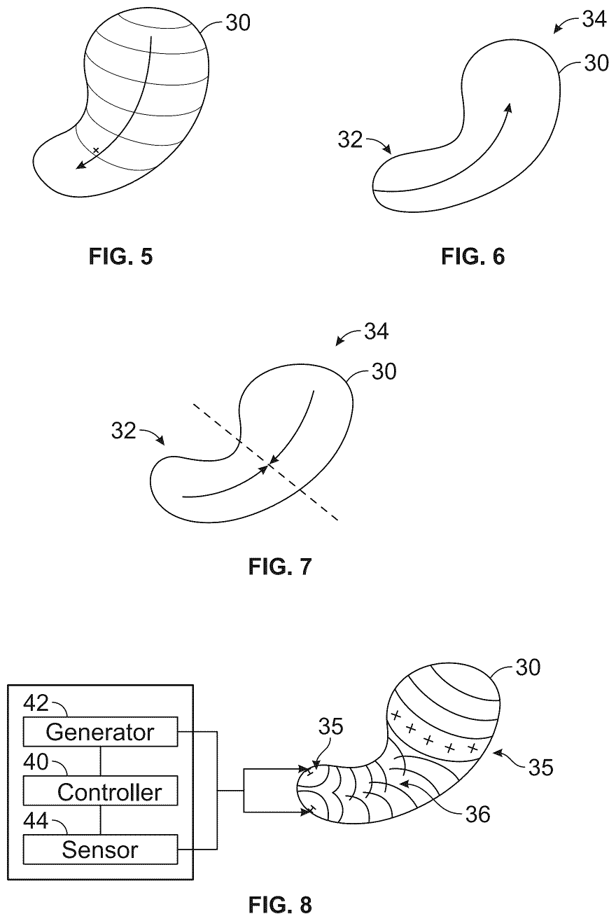

Moreover, with reference to FIG. 5, waves of electro-mechanical energy (represented as arrow) spread across the uterus 30 to create an effective contraction to facilitate passage of the fetus through and out of the birth canal. The muscles of the uterus 30 are interconnected via gap junctions between the cell walls. As electrical waves spread across the body of the uterus 30, the region being depolarized contracts and holds, and the relaxed region is associated with repolarization and stabilization. Devices can be employed to inhibit, control, change or otherwise affect this propagation of energy across the uterus in order to stall labor. A non-pharmaceutical or less pharmaceutical reliant system and method can be employed to limit or avoid side effects associated with the use of pharmaceuticals. Moreover, approaches to one or more of neuromodulation of sympathetic and parasympathetic signals can be employed to decrease contractions.

To inhibit or block neuromuscular conduction or contraction of the uterus, one or more of a constant or variable frequency energy can be employed. Additionally or alternatively, an approach involving sustaining depolarization across a mass of tissue, or using other energy which invokes relaxation of muscle can be used to inhibit or block conduction or contraction. In this regard, as shown in FIGS. 6-7, methods and devices can be used to reverse the direction of contraction of the uterus (as depicted by arrows) so that it propagates from the isthmus 32 of the uterus to the fundus 34 (FIG. 6), rather than from the fundus 34 to the isthmus 32 (as depicted by arrow; FIG. 5). Additionally, methods and devices can be utilized to pace a counter contraction against a forward contraction to thereby blunt a normal contraction (as depicted by arrows; FIG. 7). Another way to affect a contraction of the uterus 30 is to send a signal which defeats the strain receptors and decreases the reactivity of the uterus 30.

With reference to FIG. 8, the basic concept of affecting the contraction of the uterus 30 involves installing one or more electrodes 35 on or into the tissues of one or more of the cervix or uterus 30 to deliver a series of waves 36 of electro and/or mechanical stimulus to disrupt the effectiveness of the uterine electromechanical wave, to thereby render contractions ineffective or to cause fibrillations of the uterus 30 The electrodes are in communication with and are controlled by a controller 40. This communication can be provided through an electrical conduit directly or indirectly connected to the controller 40, or the electrodes can include microtechnology and a battery cooperating to provide telemetric communication wirelessly with the controller 40.

In certain embodiments, electrical stimulation of the uterus or inhibition of contractions of the uterus can be enhanced, augmented or improved with the introduction of systemic drugs. In particular, lower doses of already established tocolytics or dosages that are not generally effective on their own can be used in conjunction with electrical stimulation or the inhibition of contractions of the uterus to achieve desired results. For example, drugs such as Prostaglandin inhibitors (e.g. indomethacin, ketorolac), Calcium channel blockers (e.g., nifedipine), Nitrates (e.g. nitroglycerine) or Oxytocin receptor blockers (e.g. atosiban) can be utilized in this combined treatment approach. These drugs can be administered before, in between and/or after uterine therapy sessions or treatments. These drugs can also be locally delivered in an alternative approach.

It is recognized that a lax portion of a uterus may elongate while a contracted portion does not. In certain approaches, it is desirable to control where such elongation occurs and it is desirable to focus quieting on an upper portion of the uterus so the uterus stretches upwards with less impact or contribution to cervical ripening due to pressures generated by the fetus. Thus, a treatment device can be configured to rotate which regions of the uterus that are kept quiet to allow some number of contractions, and not keep each region of the uterus completely free of contractions. Further, the uterine treatment device or system can be configured to track and control which regions that are allowed to contract such that there is a greater ratio when the lower/cervical regions of the uterus contract while the upper regions are kept quiet, so that any stretching occurs away from the cervix and does not contribute to cervical ripening. In this way, there is control as which sections of the uterus are inhibited and which are not inhibited to thereby allow cycling of contracting tissue to allow time for recovery or other benefits. The treatment device can also be provided with various profiles of electrical inhibition to achieve desired results or results customized for a particular patient.

In one or more or each of the disclosed approaches or embodiments, the controller 40 includes a program specific to uterine treatment and is used in conjunction with electronic and mechanical devices. The controller 40 further comprises or includes a non-transitory computer-readable storage medium and a computer-program mechanism embedded therein to control or communicate with one or more of a generator 42 and a sensor 44. The controller 40 can analyze data collected by the sensor 44 in real time in order to be responsive to the condition of the uterus 30, fetus or mother, and an affect created on the uterus 30 by the energy waves provided by the generator 42. In this way, a treatment procedure can be modified as necessary to create a desired effect in the uterus 30. Here, the treatment system can learn via AI/machine learning to understand sensor readings to then potentially suggest or auto-initiate the treatment device.

As stated, the system can alternatively or additionally include structure and functionality that allows for a non-sensor approach that runs based on user input or based on a pre-programmed setting.

Electrodes or other energy generating structure 35 can be configured to apply uterine contraction-affecting energy to discrete treatment points or locations of the uterus 30 in a treatment regimen. Such electrodes 35 can be individually implanted or otherwise associated with the uterus 30, or can form a connected or disconnected array of energy providing devices. Moreover, the electrodes 35 can be placed taking a transvaginal or a percutaneous or vascular approach to target areas associated with the uterus 30.

In certain alternate embodiments, the electrodes can be placed to stimulate uterine contractions. For example, uterine contractions can be stimulated after the gestational age of a fetus is extended to full term or otherwise as desired, or if active labor progresses slowly after starting. Moreover, contractions can be stimulated where there is trouble controlling bleeding after delivery.

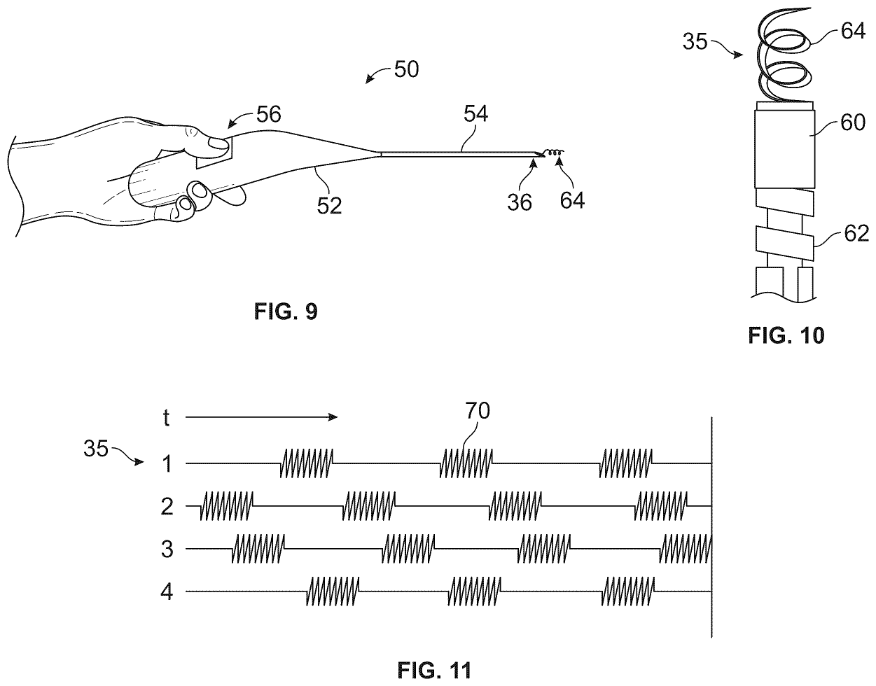

In one approach, electrodes 35 can be place using an electrode delivery apparatus 50 (See FIGS. 9-10). In one embodiment, the electrode delivery apparatus 50 includes a handle assembly 52 sized and shaped to fit comfortably and conveniently in the hand of a user. Extending longitudinally from the handle assembly 52 is an elongate shaft 54 sized and shaped to be able to reach an electrode 35 placement site from outside a patient's body. In this regard, ultrasound can be employed to determine the necessary length of the shaft 54 and/or determine the desired depth for the electrode. The operator can then select a delivery apparatus having the proper length or its length can be adjustable as dictated by the needs associated with a specific treatment.

The location of the delivery apparatus 50 tip (or needle tip, wire or catheter tip that may be utilized in a procedure) can be tracked by various means for positional feedback and to ensure safe passage and delivery. Ultrasound imaging may be utilized to ensure there are not structures in the path and for accuracy of placement. Other means may include light at the end or along the device to allow transillumination through the skin or an electromagnetic sensor within the device that can be tracked/positioned in real space and time such as surgical navigation.

One or more or a series of electrodes 35 can be loaded within the elongate shaft 54 of the delivery apparatus 50. Each electrode 35 can further be equipped with attachment structure or means for engaging the electrode at the placement site. The handle assembly further includes a deployment trigger 56 that is operatively associated with structure extending within the elongate shaft 54 to individually advance an electrode 35 out of the shaft 54 and delivery the electrode 35 at the placement site.

In one or more embodiments, a delivery apparatus can accommodate a string or column of multiple electrodes. The delivery apparatus can thus deliver multiple electrodes at a treatment site so that one stick or one approach results in the delivery of multiple electrodes. In other embodiments, a delivery apparatus is configured to deliver multiple discrete electrodes. Such electrodes can be arranged in a cartridge, and/or additional electrodes can be loaded into the delivery apparatus as needed. In this way, multiple or discrete electrodes can be delivered through one stick or approach, or electrodes can be delivered through multiple sticks or approaches.

With reference to FIG. 10, in one embodiment, the electrode 35 defines an assembly including an electrode body 60 including grooves 62, and a corkscrew or helical attachment structure 64 extending distally and longitudinally from the electrode body 60. The delivery apparatus 50 includes complementary structure for receiving the electrode assembly 35 and for causing the electrode assembly 35 to be rotated out of a terminal end of the delivery apparatus 50, and into the placement site. The attachment structure 64 is thus rotated into tissue at the placement site and forms a secure connection to the tissue.

The structure and mechanism to attach the electrode 35 at the placement site, and the electrode 35 itself can assume various alternative forms to achieve desired secure placement of the electrode 35. Accordingly, in addition to rotating an electrode 35 out of a delivery system, the delivery system can be configured to deliver an electrode 35 in a linear fashion. Additionally, the electrode 35 can embody one or more of a clamp assembly, a hook or hooks, barbs, adhesives or other attachment structure, or a frictional engagement can be relied upon for affixing the electrode 35 in place. In each approach, the delivery system would include complementary structure or functionality to accomplish delivering and positioning the electrode 35 at the placement site. Further, such approaches can also include a means for releasing or unlocking the electrode 35 from engagement at the placement site when desired.

In one or more alternative approaches, there can be provided a robotic system for the delivery and placement of electrodes or other delivery structure as desired relative to the uterus. A robotic or other guidance instrumentation can be configured to delivery devices or leads such as transvaginally or through the bladder, and can alternatively or additionally incorporate various cooperating imaging modalities.

As stated, various approaches can be employed to inhibit or block or otherwise affect neuromuscular conduction or contraction of the uterus. That is, high or variable frequency energy, sustained depolarization, or other energy which accomplishes relaxation of muscle can be employed.

Also, it may be desirable to incapacitate portions of the uterus to prevent contractions in those sections such that as contractions propagate, they are effectively stopped when they run into a section of the myometrium that is non-conductive or in other ways not capable of contracting. These sections may be entirely unable to conduct/contract or may be much less efficient at conducting/contracting. These sections may be analogous to walls or other structured barriers or may be randomly placed with the intended effect of reducing the overall contraction of the uterus. In some ways, these may be analogous to seawalls or breakwaters or other structures that block or weaken or dissipate ocean waves. It is also analogous to cardiac Maze procedures and renal denervation procedures.

Incapacitating the myometrium can take many forms in addition to electrical stimulation/inhibition of contractions. It may be desirable to use electrical, chemical, hormonal, pharmaceutical, and/or temperature (hot or cold) to affect the myometrium. For example, it may be desirable to use RF energy to ablate the myometrium along a line. The ablation may be transmural to completely separate conductive/contractile sections of the uterus. It may be desirable for safety reasons to not deliver energy completely through the myometrium and have a partial depth ablation, to weaken passage of contractile waves.

Additionally, it may be desirable to permanently affect some of the myometrial cells. It may also be desirable to use pharmaceutical or other means to provide a temporary effect which can be reversable over some time frame that may range from seconds to months. It may be acceptable for the effect to quickly end and the user can apply the treatment repeatedly or the effect may last well through the birth of the child and through the postpartum period. Examples of this may be the delivery of heat, cold, rf energy, botulin toxin, paralytic agents, muscle relaxants, inhaled anesthetics, other anesthetics agents such as nitric oxide, other relaxant or contractile agents, and other agents.

Further, it may be desirable to insert one or more drug delivery catheters that have the ability to delivery desired agents around parts or all of the uterus. Local delivery of agents may include calcium blockers, anti-inflammatory agents, progesterone, botulin toxin, paralytic agents, muscle relaxants, inhaled or other anesthetics such as nitric oxide, etc. whose effects can range from preventing signal conduction to relaxing the smooth muscle cells. These may be place/tunneled as described for the electrical leads.

In terms of strategy to deliver transient/temporary therapy (stimulation, drug, etc.), there can be multiple ways to approach this. For example, the stimulation/inhibition signal/waveform may be delivered continuously through the therapy window. It may be delivered intermittently (in a regular or irregular pattern/interval) allowing for the myometrium to recover and not form some sort of memory or ability to adapt to the therapy; it may be delivered on demand when the patient senses themselves the start of a contraction or other signal/sense/precursor to contraction; or it may be delivered after sensing the start or precursor to a contraction (the sensing may be integral to the delivery device or a completely separate system such as tocodynamometry (TOCO), intrauterine pressure catheter (IUPC), Electrohysterography (EHG) or Electromyometrial imaging (EMMI).

Again, the goal of the disclosed systems, devices and methods is to extend gestational age by delaying preterm labor and birth. By preventing, or weakening, or otherwise rendering ineffective overall contractions of the uterus, the disclosed devices and systems delay the progress of labor, cervical changes and subsequent birth.

As stated, there are several types of waveforms that can be used for electrical stimulation therapy, including symmetrical biphasic waveforms in connection with each of the disclosed approaches or embodiments. These waveforms have equal duration and amplitude for both the positive and negative phases of the waveform. Symmetrical biphasic waveforms are commonly used for muscle stimulation because they are efficient at producing muscle contractions but when produced out of sync of the native waveform can effectively block progression of the wave.

Asymmetrical biphasic waveforms can also be employed. These waveforms have unequal duration and amplitude for the positive and negative phases of the waveform. Asymmetrical biphasic waveforms can create regions of stasis and “fibrillation” and also render the myometrium incapable of passing forward an effective wave.

Pulsed waveforms are another option for use in affecting the uterus. Pulsed waveforms involve delivering short bursts of electrical energy with a fixed frequency and duration. Pulsed waveforms can be useful if used in specific frequencies to help maintain the myometrium is a state of refractive depolarization or when used at specific times to break an electromechanical wave.

Also, sinusoidal waveforms can be used in a treatment procedure. Sinusoidal waveforms produce a smooth, oscillating pattern of electrical energy. Sinusoidal waveforms can be helpful in creating an alternative and much less effective electromechanical pattern within the uterus.

Further, the contraction inhibition treatment system can be configured to operate with a pattern of actuation having known repeating cycles of on and off. The system can be configured to be active for a period of time (such as 30 minutes), and then off for a period of time (e.g. 5 minutes). These periods can be adjusted based on the stage of gestational age, physician judgment or other factors, or to take into account typical periods between contractions at give stages of gestational age and labor. These can be functionality incorporated into the system allowing for the user to initiate and control operation during the day and allowing the system to be in a sleep mode at night where the system is triggered more frequently to ensure any potential contractions are inhibited.

Moreover, the pattern of inhibition can be one or more of constant, timed, random or repeating patterns. In one aspect, the system can include functionality permitting random contraction inhibition with both on and off periods of activity, with limits placed on how long any period can be. In various other aspects, the treatment device can be initiated or triggered by sensing one or more of contractions, EMG signals, EMMI signals, fetal movements, fetal heartbeat, or specific areas of uterine contractions. All such signals can be captured and utilize AI/machine learning to better predict when a contraction or other event will occur and merits intervention.

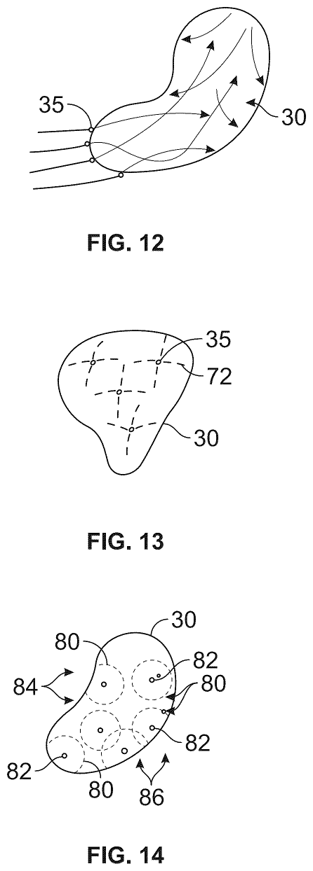

In one particular approach, with reference to FIG. 11, a plurality of electrodes 35 can be positioned at desired placement sites to provide cooperating stimulating parameters over time (t). Here, stimulation provided by pulsed waveforms 70 is designed to create a disorganized set of waves which function to break the natural electromechanical waves of the uterus during labor and cause a disorganized weak series of contractions (represented as arrows; FIG. 12). In this way, the progression of preterm labor can be inhibited or affected as desired.

Electrical inhibition of the uterus can be achieved via a combination of transcutaneously placed electrodes and transvascularly placed electrodes, or entirely transcutaneously or entirely transvascularly. Electrical inhibition can also be provided by electrodes placed on the skin alone or in combination with transcutaneously or transvascularly placed electrodes.

The electrodes can be wired or wireless. Wireless electrodes can be placed under the skin and even under the abdominal muscle and then powered via electromagnetic induction or other means. The electrodes can be placed via a transcutaneous stick or via a minor surgical procedure to access the appropriate location along the uterus. The electrode can have features that facilitate removal as desired, such as after the birth of a child. The electrode can embody smooth, tapered physical feature, and additionally or alternatively include a tag or other pull member facilitating grasping for easy removal. Removal of electrodes can be performed under some local anesthetic agent.

The electrodes can be placed in a manner to achieve overlapping of contraction inhibition effects. In this regard, the electrodes can be placed so that the effect of adjacent electrodes is less than 8-10 cm apart so that desired overlapping is achieved. Accordingly, a grid-like pattern can be used in placing electrodes to maximize an effected area of the uterus. In another approach, lines of contraction inhibition provided by electrodes can be paired from the fundus to the cervix of the uterus in a pattern similar to struts of an umbrella.

The electrical profile of stimulation provided by the electrodes can be consistent between electrodes, or can vary or be variable. In one aspect, electrical profile can have a frequency ranging from 0-60 Hz or more, and the amplitude can be 0-40 mA or more, with a pulse width from 0-50 ms or more. The pulse type can assume various shapes, for example a square wave, and the duration of a stimulation cycle can be 5 s to 60 min or more.

One access approach to the uterus is transvaginal, that is, applying electrodes to the cervix. Alternatively, a treatment approach can involve targeting the far lateral sides of the uterus that are accessible via the vagina, or by penetrating beyond the vaginal wall into the space surround the cervical base and into a deeper region of the uterus adjacent to region in proximity to the cervix. One or more electrodes could be hooked, screwed in, anchored or otherwise fixed in these locations.

The electrodes can be mono or bi-polar. If monopolar, a return electrode can be placed on the skin of the patient, similar to those used for electrosurgery. Alternatively, the electrodes can be used in pairs or combinations to deliver different focal patterns of electrical activity in the uterus. It may also be desirable to insert one or more electrodes percutaneously (possibly under ultrasound guidance) into or onto the fundus of the uterus through the abdominal wall. These could be used as the sole sources or in combination with sources placed transvaginally. Transrectal or trans-vesical placement could also be possible.

Moreover, access to the intraperitoneal or other portions of the uterine myometrium may be done transcutaneously via the abdomen of the mother. Access to the extraperitoneal portion of the uterus also may be accomplished transvaginally via the cervix.

Turning to FIG. 13, in one particular approach, a plurality of electrodes 35 can be attached to a uterus 30 percutaneously via tunneling through an abdominal entry or puncture site. The electrodes 35 themselves can embody leads 72 that spread out when the electrode 35 is placed at or about the uterus 30. Such leads 72 can further include structure for affixing the electrode 35 in place. Further, the delivery apparatus employed to deliver the electrodes 35 can embody structure that can accomplish the creation of an entry site, as well as the removal and collection of an electrode.

It may be required to place multiple electrodes, spaced a given distance apart, to ensure greater treatment coverage of the myometrium. The effective regions may overlap in effectiveness region. It may also be acceptable for the effective regions to not overlap as any contractions in between regions will be effectively blocked or prevented from propagating past regions of treatment.

In one embodiment, area coverage of the myometrium solely via the abdomen is achieved. A rough estimate is that 25-30% of the uterus is accessible via the anterior abdomen transcutaneously without tunneling. There can be sufficient coverage to access the uterus via the cervix/transvaginally. A rough estimate may be 10-20% of the inferior uterus is accessible. In another embodiment or combination, be required to combine treatment via both the abdomen and transvaginally to address enough of the myometrium to minimize contractions.

In another embodiment, access to more lateral, superior and even posterior portions of the myometrium from the abdomen is achieved. After gaining transcutaneous access, it is possible to tunnel along a plane along the peritoneum, perimetrium, myometrium with a catheter (steerable or non-steerable), guidewire (steerable or not), everting inflatable member or other device and gain access and deploy a multitude of electrodes or other devices to further regions of the uterus like the superior, lateral or posterior regions. This reduces the number of access sites/transcutaneous sticks required to access all the desired locations as multiple electrodes or treatment sites can be located along a given track along the uterus.

With reference to FIG. 14, in one approach, there is shown a uterus 30 with electrodes affixed thereto. A treatment region or region of effect 80 is associated with each treatment point 82. Such treatment regions 80 can define either non-overlapping regions 84 and/or regions that overlap 86 with other treatment regions 80 to create the desired effect on the uterus 30.

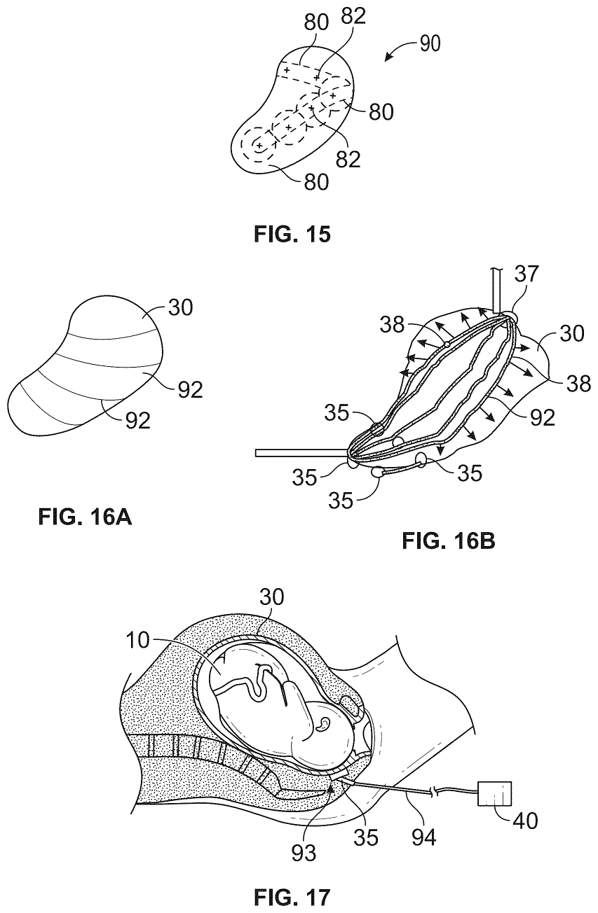

In another approach (FIG. 15), a plurality of electrodes can be inserted through a single access site 90 to the uterus 30. Through the access site 90, a path of tunnelling is created to attach or associate the electrodes with the uterus 30 at discrete treatment locations 82. Here also, each treatment location 82 has a treatment region of effect 80 that can either be overlapping or non-overlapping to have the desired effect on the uterus 30.

As shown in FIGS. 16A-B, various lines of treatment 92 can be created to have the desired effect on the uterus 30. In one particular approach (FIG. 16B), one or more electrodes 35 are placed at the cervix or circa-cervix, or at the edges of the uterus 30. The electrodes 35 can be percutaneously or transvaginally delivered onto the cervix 30 or into muscle just below where there is electrical access to the one or more regions of the uterus. At least one return electrode 37 is inserted into or onto the fundus transcutaneously superiorly as possible so that an electrical trans-body stimulus is created.

An electrical source is applied to provide enough energy to create a fibrillation-like response locally in the uterus 30 to thereby stall the uterus 30 from being functional. Waves of energy 38 propagate from positive to negative terminals and course across the uterus and gap junctions and fluid connections that exists in uterine tissue in a pattern fanning out modestly, but primarily across paths of least resistance.

In this way, there is created a source for a stimulus that prevents tissue from re-polarizing sufficiently. At the edge of the wave 38, there will be some propagation and that effect the tissue as much as a distance of 8-10 cm. However, the uncovered regions can be minimized by having multiple electrodes at the source to create a pattern of energy propagation in multiple regions anteriorly and posteriorly as they course around uterus to the return electrode 37. A single electrode or multiple electrodes 35 can fire simultaneously to create the same wave at the same time or alternatively, the electrodes can fire in sequence. Where the electrodes are coordinated to fire at the same time, there is created a uniform stimulus across the uterus 30.

Referring now to FIGS. 17-21, there are presented various additional approaches to affecting or inhibiting contractions of a uterus 30. In each approach and for those described above, stimulus provided by electrodes 35 or other energy delivery apparatus can be responsive to one or more of fetus heart rate, pulse oxygen levels, uterine contractions or electrical activity, or physiology or conditions (i.e. heart rate, pain) associated with the mother. Stimulation provided by the electrodes 35 can be continuous to maintain cellular depolarization, or intermittent or modulated in response to uterine, fetal or mother conditions.

In a first alternative approach to treatment (FIG. 17), an electrode or an array of electrodes 35 are implanted within an intraabdominal space such as the pouch of Douglas 93, which is a potential space in the female human body located between the uterus 30 and rectum or anterior cul-de-sac, which is a potential space in the female human body located between the uterus 30 and bladder. In this and each of the disclosed embodiments, the array of electrodes 35 can embody a mesh or a patch structure, and can be affixed in place using the structure or approaches presented above, namely, via a helical or corkscrew projection, hooks or barbs, using clamp structure, adhesive, or friction. Moreover, as described above, the electrodes 35 can include structure or functionality that permits their deployment by a delivery apparatus as well as facilitate easy removal.

While an array of electrodes 35 can be directly connected to a controller via an electrical cable 94, in each of the disclosed embodiments, a wireless approach can be taken to communicate with the controller 40. Wireless communication can be BLE, wifi or via other wireless means to communicate to a remote control/actuation device for the patient or other health care provider to initiate the inhibition. The controller 40 can also be embodied in a box or other structure that is worn on the abdomen, waist, or leg of a patient, or it can reside remotely to allow patient mobility so that they are not constrained to bedrest. There can be a button or other actuation method provided physically on the controller 40 to allow for patient activation.

Also, in each of the disclosed embodiments, inhibition settings and patterns may need to change over time in order to respond to the changing uterus. For example, the conductivity between myometrial cells changes with the increase in number of gap junctions as gestational age increases, and especially during labor. The treatment system is thus configured to be able to change frequency, amplitude and pulse width over time or based upon measurements or sensor reading.

Similarly, as gestational age increases, the disclosed uterine treatment systems can change the timing of when inhibition is on or off, the interval between being on or off, and which treatment regions of the uterus are turned on or off. This can be due to expected intervals between contractions, expected intensity of contractions or the like. This can also be driven by the time of day or when the patient is awake, asleep, resting or active. Further, the mechanism for triggering of the treatment system can change over time. That is, the treatment system can manually trigger earlier or when contractions are further apart or can be triggered constantly on/off or following a pattern to maximize time as contractions continue and are closer together or at a later gestational age. The triggering mechanism can also be different when the patient is sleeping or awake.

Additionally, a percutaneous approach to implanting the electrode array 35 at the pouch of Douglas 93 can be taken, or alternative approaches such as transvaginal approach can be employed. The pouch of Douglas 93 is useful for the implantation of an array of electrodes 35 as it is adjacent the uterus 30 and can accommodate the electrode structure to thus create a desired effect on the contractions of a uterus 30.

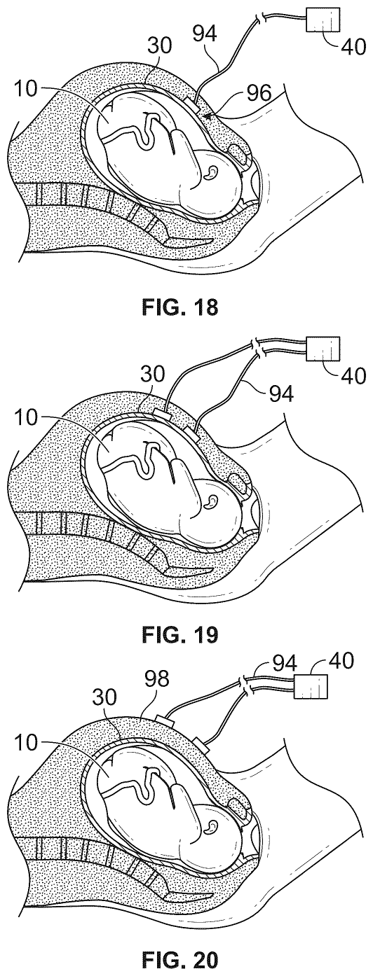

As shown in FIG. 18, using one or more of the approaches and devices described above, an electrode or an array of electrodes 35 can be implanted to a pre-peritoneal space 96. Here, there may be a need for balloon dissection of the space to allow for the delivery of the electrode array 35. Alternatively, as shown in FIG. 19, one or more electrodes or array of electrodes 35 can be attached to or adjacent an outer surface of the uterus 30. In a related approach (not shown), a space between the amniotic sac and an internal wall of the uterus can be developed for the placement of one or more electrodes. In each of these approaches, the electrodes 35 are configured to have affect or inhibit the contractions of the uterus 30 by delivering a series of waves of electro and/or mechanical stimulus to disrupt the effectiveness of the uterine electromechanical wave, to thereby render contractions ineffective or to cause fibrillations of the uterus 30.

Further, one or more electrodes or an array of electrodes 35 can be affixed to the outer skin 98 of a patient (FIG. 20). Here, the electrodes 35 can embody pads that provide a transabdominal, non-invasive or defibrillation approach to treatment. In one particular aspect, such treatment involves pelvic nerve modulation. Monophasic or biphasic waveforms can be utilized to create the desired effect in the uterus 30. Further, energy delivery timing can be based on measured electrical activity of or alternatively, pressure associated with the uterus 30. Such timing also can be dependent or guided by the fetal heartrate or other signals relevant to the health or condition of the fetus or mother. The response to such data can be automated and controlled by the controller 40, or responsive to the data can be manual.

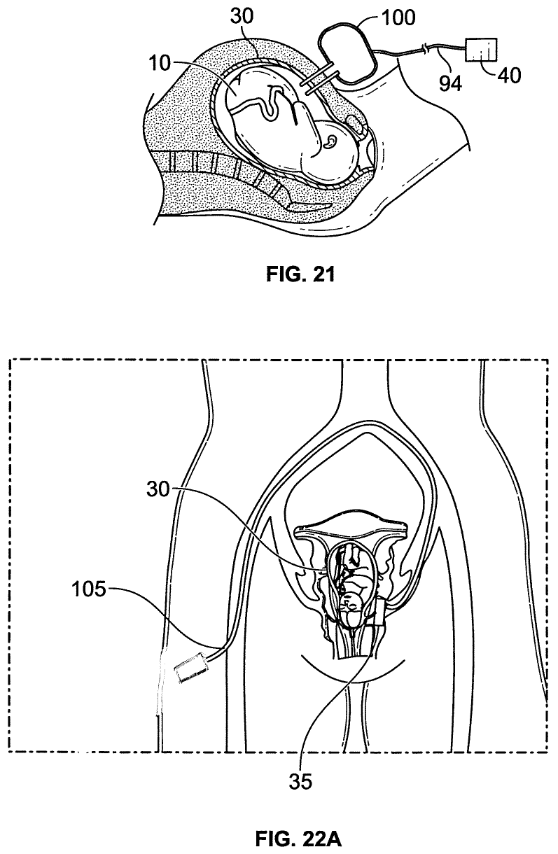

With reference to FIG. 21, in another aspect, to affect or inhibit the contractions of the uterus 30 there is provided a pressure relief bladder 100. The pressure relief bladder 100 can operate independently or be controlled by a controller 40. The bladder 100 is configured to allow the flow of amniotic fluid out of the uterus 30 during contractions in order to reduce wall strain and smooth muscle actuation of the uterus 20. The bladder 100 can include a valve (not shown) that is one-way, active or adjustable, or controlled by a controller in response to uterine, fetal or mother activity or conditions. Further, while shown configured in a transabdominal configuration, the bladder can also be placed intraabdominally or transvaginally.

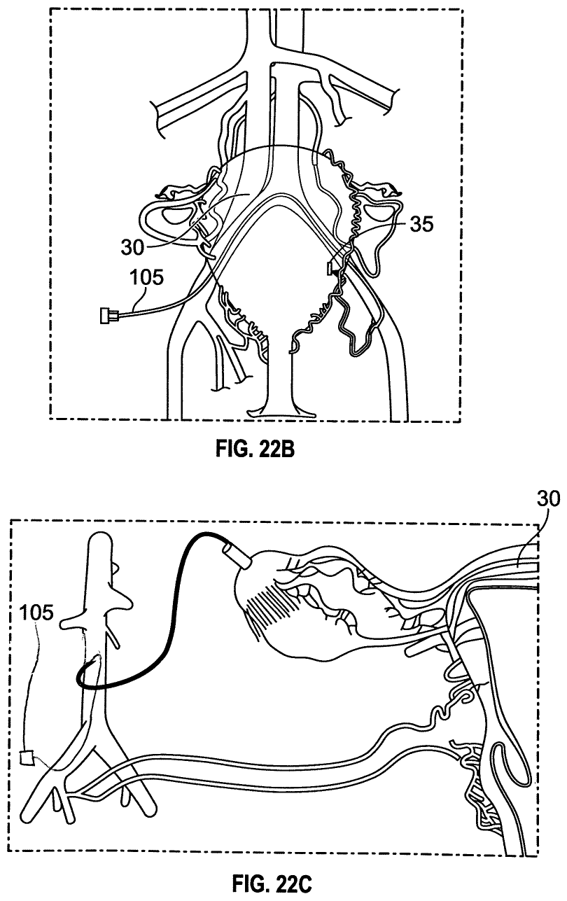

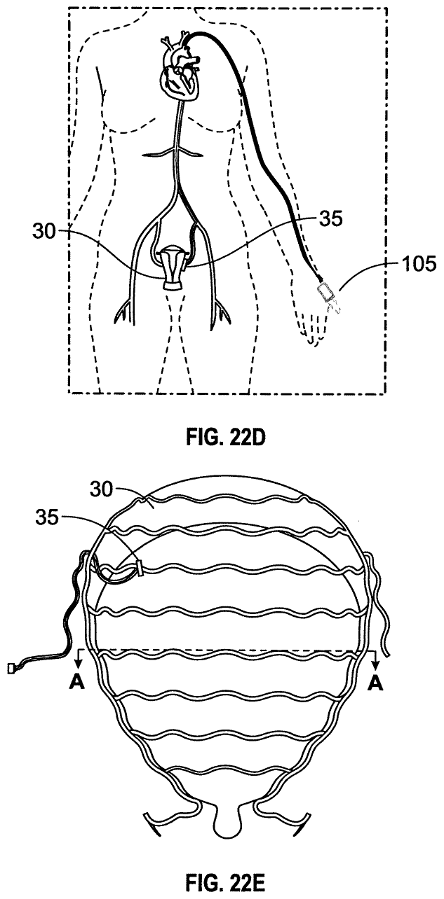

As shown in FIGS. 22A-E, in yet other embodiments, a transvascular approach is utilized to place or implant electrodes 35 at or adjacent the uterus 30. In this regard, uterine vasculature including the uterine arteries or veins (left and/or right) 102 are used to deliver the electrodes 35. Drugs or other agents or agents can also be delivered via uterine vasculature, where such drugs or agents can be delivered via a bolus, intermittently or via continuous infusion.

To gain access to this vasculature, a Seldinger technique is used to enter the femoral artery, vein, lymphatic or other vasculature that eventually leads to the uterine vasculature. With reference to FIG. 22B, one preferred approach to gaining access to the uterine artery is via the internal iliac artery or via a branch or division of the internal iliac artery. It is also possible to access an ovarian artery (via the aorta) to gain access to the uterus (FIG. 22C), or by way of other vasculature reachable therefrom. Also, a radial approach can be used to gain access to either ovarian or uterine vessels (FIG. 22D).

Further, arcuate or radial arteries or veins can be used to access myometrium (FIG. 22E). The arcuate artery generally runs on the surface of the uterus 30 while the radial arteries penetrate the myometrium. The arcuate vessels tend to encircle the uterus by coursing within the myometrium just beneath its serosal (outer) surface and provide useful access to the uterus 30.

With reference again to FIG. 22A, a preferred path to access can be via the femoral approach through the internal iliac to the uterine artery, and then into the arcuate arteries for targeted regions of the uterus 30 using both the left and right sides. Once at the desired site, a catheter 105 can be employed to advance a needle or other penetrating member (not shown) that facilitates the advancement and delivery of an electrode 35 or a series of electrodes 35 into the myometrium. This can also be along a line extending further from a distal end of the catheter 105.

The catheter 105 can also be used to advance a needle (not shown) or other catheter structure that penetrates out of the vessel and into the myometrium or desired target tissue. This structure can deliver drugs or other agents to selected sites. It may also be desirable to advance a catheter 105 from wherein the needle to travel further along the myometrium to deliver an agent beyond the catheter path. It can also be used to advance and leave a catheter for continuous or intermittent infusion of an agent.

It is also possible to deliver along a multitude of directions from a given location by changing the angle from which the penetrating member exits the catheter 105. This can be by using a steerable member or a curved needle that changes vector as it is pushed out. Once a trajectory is established, another catheter, wire or other member (not shown) can be extended from the needle (not shown) along a straight path. In another approach, the catheter 105 can be deflectable and configured to allow advancement of an electrode assembly/wire or other member to the desired target location.

Electrodes 35 can be deposited and then communicate or be powered or induced electromagnetically from an external device that is placed on the body. Alternatively, the electrodes 35 can be wired and include leads that exit from the vasculature and be connected to a controlling or powering unit.

Further, the electrical leads 94 attached to the electrodes 35 can reside in the uterine vasculature and be attached to a controller 40. Alternatively, a wireless approach to control of the implanted electrodes 35 can be employed. Again, the electrodes 35 are placed to affect or inhibit the contractions of the uterus 30 as dictated by a treatment procedure.

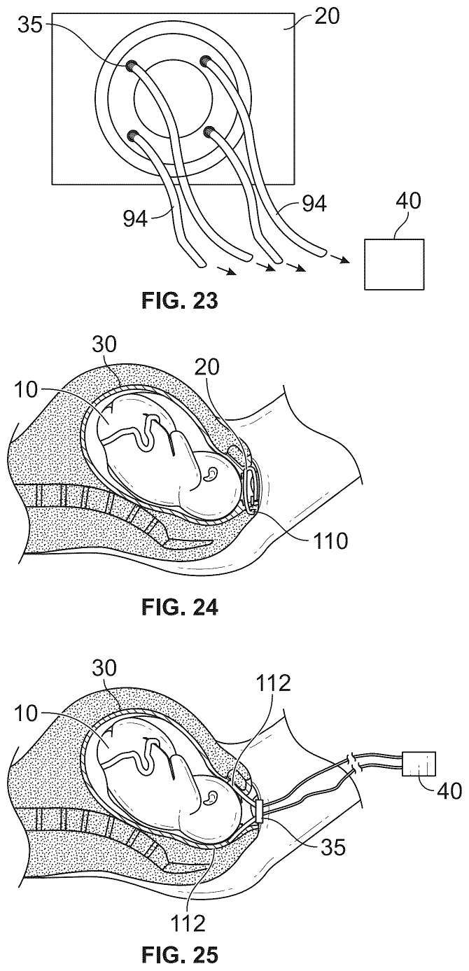

As stated, transvaginal approaches can be employed to implant structure to affect the contractions of a uterus. As shown in FIG. 23, a plurality of electrodes 35 can be implanted directly to the cervix 20. Leads that extend from each of the electrodes 35 can extend to one or more of a controller 40 that controls stimulation in response to a detection system, or the leads 94 can extend to discrete devices providing such functionality. In a related approach (FIG. 24), the electrodes are carried by a self-contained ring 110 that fits about the cervix 20. The electrode array is configured for both sensing and stimulation so that contractions of the uterus 30 can be affected or inhibited. Alternatively, as shown in FIG. 25, leads 112 extending from an electrode array 35 are tunneled into the uterine walls to provide the desired electrical waves in the uterus 30. Here, the electrodes 35 can be placed intravaginally or extravaginally.

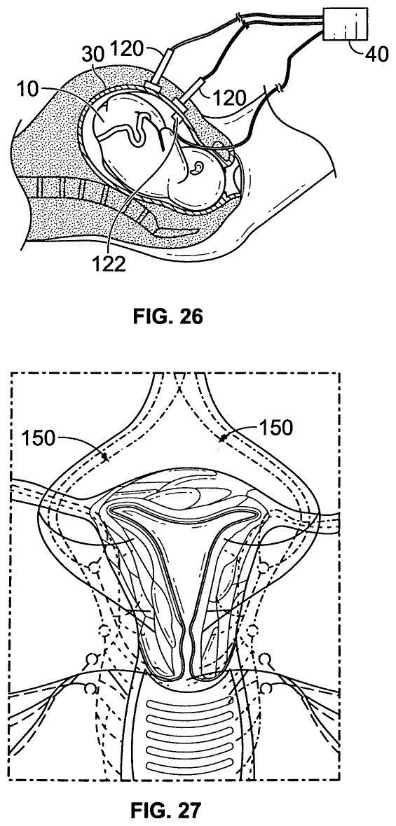

Referring now to FIG. 26, in yet another approach to treatment, portions of the uterus can be ablated, such as in a Maze procedure, to have the desired effect on uterine contractions as well as the ability of a uterus to contract. A variety of ablation patterns can be used including circumferential, anterior only, or partial or full tissue thickness ablation. Also, an ablation pattern that directs depolarization towards a least contractile region of the uterus can be used, or one that focuses on the isolation of a pace making region of the uterus. Mechanisms or devices 120 providing RF or other energy or temperature generating energy are placed on the uterus 30 to create the desired pattern of ablation. Ablation devices can be linear to burn lines along muscle or embody rigid devices which are dragged along the uterine surface. A unipolar or a bipolar arrangement can be used to provide the ablation energy. In this regard, a transvaginal cooperating component 122 can placed within the uterus 30 to provide necessary ablation functionality in a monopolar arrangement. Here also, a transvascular approach to the uterus 30 can be taken as described above to implant or position ablation devices. Further, the ablation devices can be connected to a controller 140 so that ablation can be responsive to the condition of one or more of the uterus 30, the fetus or mother.

With reference to FIG. 27, in alternative approaches, electromagnetic fields, focused or high energy ultrasound, monopolar or bipolar RF energy, transcutaneous electrical stimulation, thermo-acoustic waves, local pharmacological agents, or systemic pharmacological agents can be employed to create a desirable electrical stimulus both invasively and non-invasively to modulate the nervous system remote to the uterus in order to signal and modulate the myometrium to reduce electrical signals and/or muscular contractions. Accordingly, electrodes (not shown) can be implanted or placed outside a patient's body and positioned as needed relative to nerves 150 communicating with the uterus and cervix to create a desired effect on the uterus to inhibit pre-term contractions. A wireless or direct connection with a controller can be provided to control the operation of the electrodes 35 in response to the condition of the uterus and/or baby, automatically, pursuant to a pre-determined regimen, or as directed by the patient or healthcare provider.

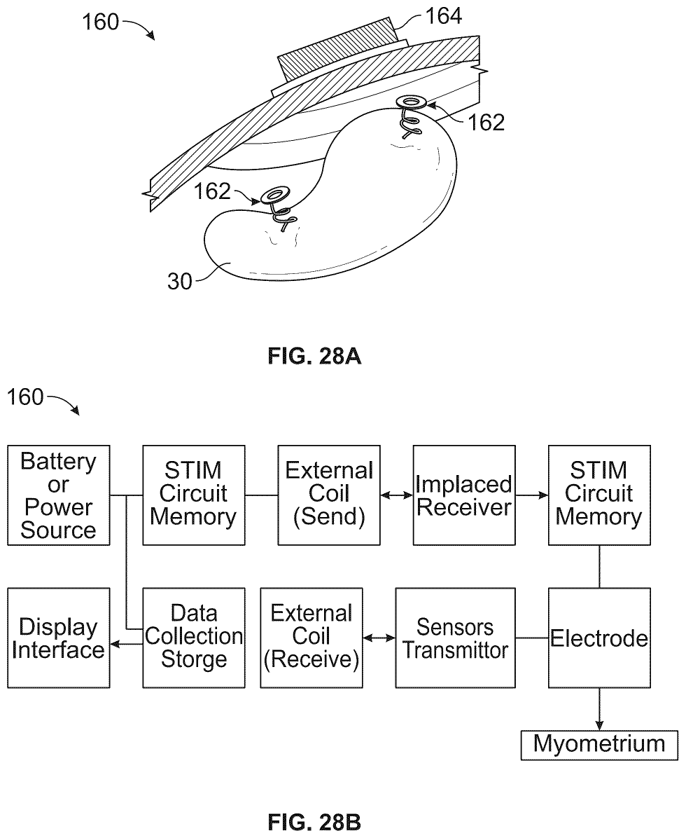

In the event that long term stimulation of the uterus 30 is necessary (See FIGS. 28A-B), it can be desirable to have a leadless system 160 including one or more receiver stimulators 162 that allows intramuscular stimulation but couples the energy from an external source 164. Power from the external source 164 can be ultrasound, electromagnetic, RF or mechano-acoustic. The receiver stimulator 162 collects this energy and using implanted on-board circuitry, translates this energy into a stimulation wave as pre-set by the user. The stimulation waveforms can also be adjusted remotely and/or in real time. The leadless system can be further configured to collect data from the electrodes or from on-board accelerometers to provide feedback to the user as to how best adjust therapy.

In one particular approach (FIG. 28B), the leadless system 160 embodies a two-way communication and control network. The leadless system 160 includes an external battery or power source that supports stimulation circuitry/memory and data collection/storage functionality and communicates with a display or interface. The external power source further includes coils that are configured to communicate with implanted receiver stimulators. An external power source coil is configured with send functionality that is configured to have two-way communication with an implanted receiver that is in communication with stimulation circuitry and memory that communicates with an electrode of the implanted stimulator. An external power source coil is also configured to have two-way communication with sensors/transmitters of the implanted stimulator, the sensors/transmitter also being in communication with the electrode. This arrangement thus provides desired and controlled long term stimulation of the uterus and can be observed or controlled through the display or user interface.

Accordingly, various approaches to extending gestational age by delaying birth are presented. The disclosed approaches are configured to provide an effective and focused approach to affect or inhibit the contractions of the uterus.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the present disclosure.

That which is claimed is:

1. A system for affecting or controlling the electrical communications across a uterus, comprising:
an energy means for disrupting or suppressing a contraction wave of the myometrium and rendering the contraction wave ineffective or blocking the contraction wave entirely to thereby delay birth.

2. The system of claim 1, further comprising one or more electrodes configured to be placed adjacent or implanted within the uterus.

3. The system of claim 2, wherein the one or more electrodes are configured to deliver a series of electro or mechanical stimulus to disrupt an effectiveness of uterine contractions.

4. The system of claim 2, further comprising a controller configured to communicate and control operation of the one or more electrodes.

5. The system of claim 1, further comprising a monitoring means configured to monitor an affect created on the uterus.

6. The system of claim 1, further comprising a sensing means configured to sense one or more of a condition of the uterus, a fetus or a mother.

7. The system of claim 1, wherein an electrical stimulus is provided to cause depolarization and repolarization of smooth muscles of the uterus.

8. The system of claim 7, wherein electrical stimulus is automatically responsive to sensed changes in the uterus.

9. The system of claim 8, wherein the electrical stimulus is pre-programed based on empirical data collected in advance of a treatment procedure.

10. The system of claim 2, further comprising a delivery apparatus configured to contain and deploy the one or more electrodes at a placement site.

11. The system of claim 2, wherein the one or more electrodes are spaced to ensure a desired treatment coverage of the uterus.

12. The system of claim 2, wherein the one or more electrodes are placed to have one or more of overlapping effectiveness regions or non-overlapping effectiveness regions.

13. The system of claim 1, further comprising structure and functionality to ablate selected sections of the uterus.

14. The system of claim 2, wherein the one or more electrodes are placed in a peritoneal space.

15. The system of claim 2, wherein the one or more electrodes are placed on an outer surface of the uterus.

16. The system of claim 2, wherein the one or more electrodes are placed within a space between an amniotic sac and an internal wall of the uterus.

17. The system of claim 2, wherein the one or more electrodes are implanted within the cervix.

18. The system of claim 2, wherein the one or more electrodes are placed within walls of the uterus.

19. The system of claim 1, wherein tissue ablation is performed to alter electromechanical conduction.

20. The system of claim 1, wherein the energy means is configured to provide long term disruption or suppression of contraction waves of the uterus and render the contraction waves ineffective or block them entirely to thereby delay birth.
